(19) United States
(12) Patent Application Publication
(10) Pub. No.: US 2026/0301896 A1
(43) Pub. Date: Oct. 1, 2026

(54) MEDICAL INFORMATION PROCESSING DEVICE, METHOD, AND STORAGE MEDIUM

(71) Applicant: NEC Corporation, Tokyo (JP)

(72) Inventors: Daisaku SHIBATA, Tokyo (JP); Masanori Tsujikawa, Tokyo (JP); Yutaka Uno, Tokyo (JP); Kunikiko Sadamasa, Tokyo (JP); Riko Irikura, Tokyo (JP); Hayato Ota, Tokyo (JP); Akira Yamauchi, Tokyo (JP); Mayu Kaya, Tokyo (JP); Tsugumi Okada, Tokyo (JP); Yasunari Takei, Tokyo (JP)

(73) Assignee: NEC Corporation, Tokyo (JP)

(21) Appl. No.: 19/574,593

(22) Filed: Mar. 23, 2026

(30) Foreign Application Priority Data
Mar. 28, 2025 (JP) 2025-054897

(51) Int. Cl. G16H 10/60

(52) U.S. Cl. CPC G16H 10/60

(57) ABSTRACT

A medical information processing device 1X includes a medical entity acquisition unit 15X, a text acquisition unit 16X, a prompt generation unit 17X, and a medical document generation unit 18X. The medical entity acquisition unit 15X acquires a medical entity included in a medical record. The text acquisition unit 16X acquires text corresponding to the medical entity from the medical record. The prompt generation unit 17X generates a prompt for instructing a language model to generate a medical document, the prompt including the medical entity and the text, wherein the language model is trained to output an answer to a prompt upon receiving the prompt. The medical document generation unit 18X generates the medical document based on the prompt and the language model.

### INCORPORATION BY REFERENCE

This application is based upon and claims the benefit of priority from Japanese Patent Application No. 2025-054897, filed on Mar. 28, 2025, the disclosure of which is incorporated herein in its entirety by reference.

### TECHNICAL FIELD

The present disclosure relates to a technical field of a medical information processing device, a method, and a storage medium for processing medical information.

### BACKGROUND

Among various types of work performed by a medical staff, work of recording a medical practice performed by the medical staff on a patient is essential for sharing patient information with a related medical staff, proving that an objectively appropriate medical practice has been performed, and the like. On the other hand, the work related to the medical record has a large burden on the medical staff. As a technology related to generation of a medical document based on a medical record, Patent Literature 1 discloses a technology of generating a summary from medical records by a medical staff.

### Citation List

### Patent Literature

Patent Literature 1: JP 2020-38602A

### SUMMARY

The medical document can be generated with artificial intelligence (AI) by using medical entities extracted from the medical record. On the other hand, in the medical document generated in this way, a hallucination may occur due to lack of consideration of connections between the medical entities.

In view of the above-described problems, an object of the present disclosure is to provide a medical information processing device, a method, and a program capable of generating an accurate medical document from a medical record.

In an example aspect of the present disclosure, there is provided a medical information processing device including: a medical entity acquisition means for acquiring a medical entity included in a medical record; a text acquisition means for acquiring text corresponding to the medical entity, from the medical record; a prompt generation means for generating a first prompt for instructing a language model to generate a medical document, wherein the first prompt includes the medical entity and the text and wherein the language model is trained to output, upon receiving a prompt, an answer to the prompt; and a medical document generation means for generating the medical document based on the first prompt and the language model.

In an example aspect of the present disclosure, there is provided a method executed by a computer, including: acquiring a medical entity included in a medical record; acquiring text corresponding to the medical entity, from the medical record; generating a first prompt for instructing a language model to generate a medical document, wherein the first prompt includes the medical entity and the text and wherein the language model is trained to output, upon receiving a prompt, an answer to the prompt; and generating the medical document based on the first prompt and the language model.

In an example aspect of the present disclosure, there is provided a program executed by a computer, the program causing the computer to: acquire a medical entity included in a medical record; acquire text corresponding to the medical entity, from the medical record; generate a first prompt for instructing a language model to generate a medical document, wherein the first prompt includes the medical entity and the text and wherein the language model is trained to output, upon receiving a prompt, an answer to the prompt; and generate the medical document based on the first prompt and the language model.

An example advantage according to the present disclosure is to generate an accurate medical document from a medical record.

### BRIEF DESCRIPTION OF THE DRAWINGS

FIG. 1 illustrates a schematic configuration of a medical information processing system.

FIG. 2 illustrates a hardware configuration of a medical information processing device.

FIG. 3 is an example of functional blocks of a processor of the medical information processing device.

FIG. 4 is a diagram illustrating a flow of processing executed by a medical entity acquisition unit, a medical entity selection unit, and a text acquisition unit in a specific example in which medical document generation processing is performed with a progress record as a target medical record.

FIG. 5 is a diagram illustrating a flow of processing executed by a prompt generation unit and a medical document generation unit in the specific example of the medical document generation processing.

FIG. 6 illustrates a prompt in a comparative example and a medical document generated by the prompt.

FIG. 7 illustrates an example of a flowchart executed by the medical information processing device.

FIG. 8 illustrates a schematic configuration of a medical information processing system.

FIG. 9 is a block diagram of a medical information processing device.

FIG. 10 is an example of a flowchart showing a processing procedure executed by the medical information processing device.

### EXAMPLE EMBODIMENT

Hereinafter, example embodiments of a medical information processing device, a method, and a storage medium will be described with reference to the drawings.

### First Example Embodiment

### (1) System Configuration

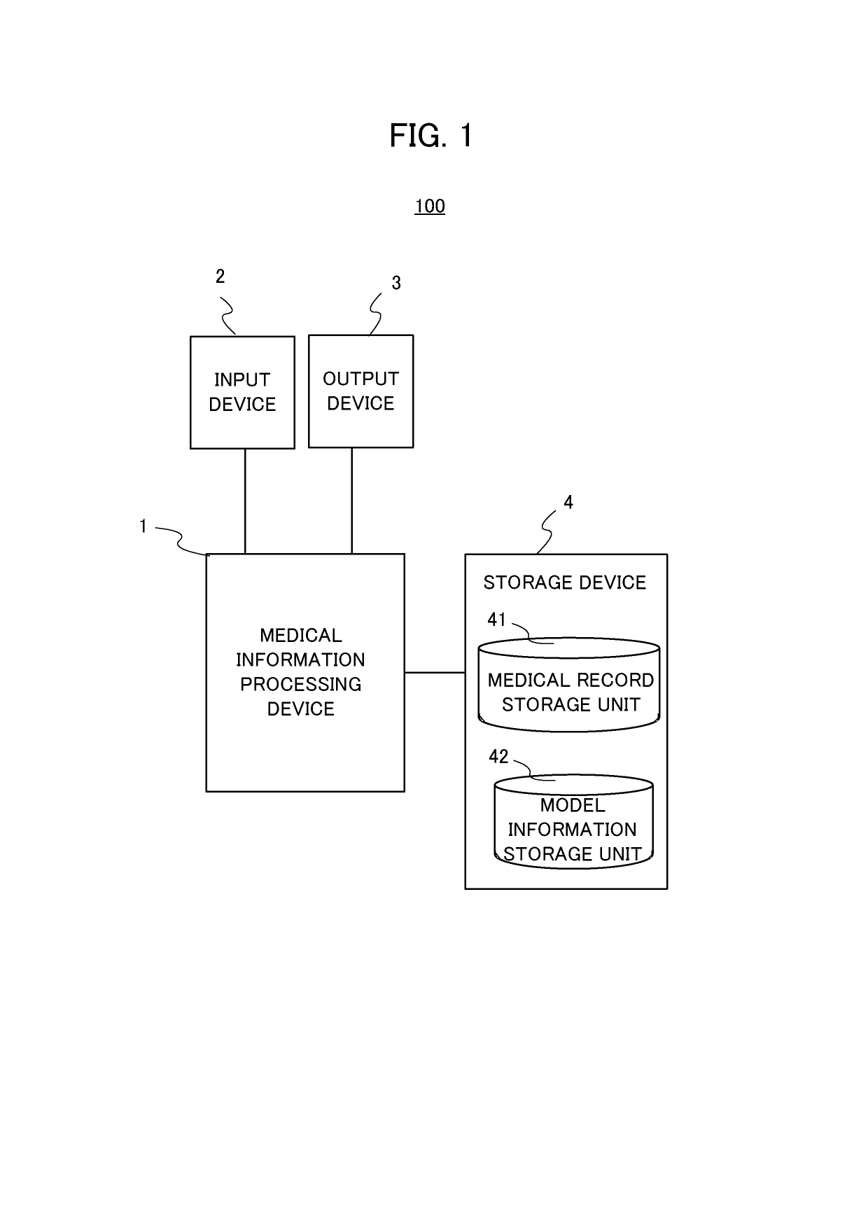

FIG. 1 illustrates a schematic configuration of a medical information processing system 100. The medical information processing system 100 generates a medical document based on a medical record of a specified patient.

Hereinafter, the “medical record” refers to various records created in a process of medical treatment for states (a physical condition, a condition of a disease, treatment to be applied, and the like) of the patient, and includes a medical record (clinical record), a prescription, an operation record, a nursing record, an examination observation record, an X-ray photograph, a referral, a pathological report, an image interpretation report, a summary of a medical treatment progress during hospitalization related to a discharged patient, and the like. Hereinafter, unless otherwise stated, the medical record is assumed to refer to electronic data in which the above-described various records are electronically stored. The medical document generated by the medical information processing system 100 from the medical record is a summary of sentences included in the medical record, and examples of such medical documents include a document (memo) describing a treatment progress, a discharge summary, a nursing summary, and the like.

The medical information processing system 100 mainly includes a medical information processing device 1, an input device 2, an output device 3, and a storage device 4.

The medical information processing device 1 performs processing related to generation of the medical document based on the medical record. The medical information processing device 1 performs data communication with the input device 2, the output device 3, and the storage device 4 via a communication network or by wireless or wired direct communication. The medical information processing device 1 identifies a medical record (also referred to as a “target medical record”) for which a medical document is generated, based on an input signal or the like supplied from the input device 2, for example. The medical information processing device 1 also generates an output signal, and supplies the generated output signal to the output device 3. The output signal in this case is, for example, display information (audio information may be included) for receiving an input necessary for generating the medical document or a correction input of the generated medical document. The medical information processing device 1 may store the generated information related to the medical document in the storage device 4, or may transmit the generated information related to the medical document to an external device (not illustrated). The generated medical document may be used for a document, for example, a referral, a medical certificate, a discharge summary, or another document that requires description of a summary of medical record, which is created by a medical staff.

The input device 2 is an interface that receives a manual input (external input) of information by a user. The input device 2 may be, for example, various user input interfaces such as a touch panel, a button, a keyboard, a mouse, and an audio input device. The input device 2 supplies a generated input signal to the medical information processing device 1. The output device 3 outputs predetermined information based on the output signal supplied from the medical information processing device 1. The output device 3 is, for example, a display, a projector, a speaker, or a printer.

The storage device 4 is a memory that stores various types of information necessary for processing executed by the medical information processing device 1. The storage device 4 functionally includes mainly a medical record storage unit 41 that stores medical records of patients and a model information storage unit 42 that stores model information necessary for execution of a machine learning model used by the medical information processing device 1.

The medical record storage unit 41 stores the medical records of the patients. Each record of the medical records stored in the medical record storage unit 41 includes, for example, identification information of the medical record, information of date and time when the medical record is created (or a medical treatment is performed), identification information of the patient, and content of a medical record (for example, a clinical record).

The model information stored in the model information storage unit 42 includes, for example, model information (configuration information) for configuring a large language model (LLM), model information for configuring a natural language understanding model to be used for natural language processing, and the like. Each piece of the model information includes, for example, various parameters of a deep learning model trained through machine learning, such as a layer structure, a neuron structure of each layer, the number of filters and a filter size in each layer, and a weight of each element of each filter.

Here, definition of the large language model or language model will be described. The language model is a machine learning model trained to learn relationships between words in sentences and generates, from an object character string, a related character string related to the object character string. By using the language model trained to learn sentences in various contexts, it is possible to generate the related character string having appropriate content related to the object character string. For example, a case where the language model is used in question answering will be described. The language model receives input of a question “What kind of country is Japan?” as a target character string. At this time, the received question is also referred to as a “prompt”. The language model generates a character string such as “Japan is an island country in the Northern Hemisphere ...” as an answer to the question. A training method of the language model is not particularly limited, but as an example, the language model may be trained in such a way as to output at least one sentence including an input character string. As a specific example, the language model may be a generative pre-trained transformer (GPT) that outputs a sentence including an input character string by predicting a character string having a high probability of following the input character string, or ChatGPT based on the GPT.

The storage device 4 may be an external storage device such as a hard disk connected to or incorporated in the medical information processing device 1, or may be a storage medium such as a flash memory. At least a part of the information stored in the storage device 4 may be stored by the medical information processing device 1. The storage device 4 may be a server device that performs data communication with the medical information processing device 1. The storage device 4 may include a plurality of devices. In this case, instead of the medical information processing device 1, the server device may execute the machine learning model (language model) based on the model information. In this case, the server device executes the machine learning model when the server device receives information (for example, a prompt) necessary for execution of the machine learning model or the like from the medical information processing device 1, and transmits information generated by the machine learning model to the medical information processing device 1.

Note that, a configuration of the medical information processing system 100 illustrated in FIG. 1 is an example, and various changes may be made to the configuration. For example, the input device 2 and the output device 3 may be integrally configured. In this case, the input device 2 and the output device 3 may be configured as a tablet terminal (including a smartphone) integrated with or separated from the medical information processing device 1. The medical information processing device 1 may include a plurality of devices. In this case, the plurality of devices forming the medical information processing device 1 exchanges information necessary for executing processing allocated in advance between the plurality of devices. In this case, the medical information processing device 1 functions as a system.

### (2) Hardware Configuration

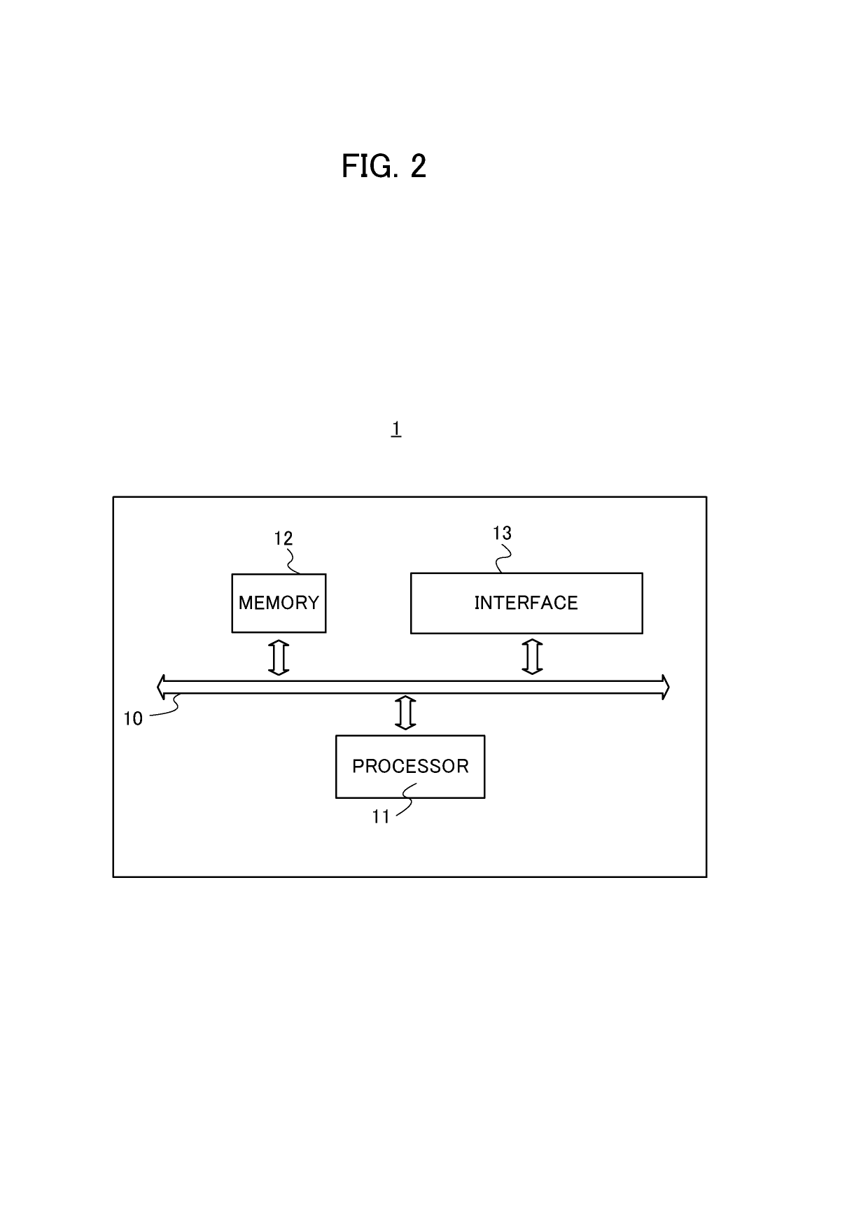

FIG. 2 illustrates a hardware configuration of the medical information processing device 1. The medical information processing device 1 includes a processor 11, a memory 12, and an interface 13 as hardware. The processor 11, the memory 12, and the interface 13 are connected via a data bus 10.

The processor 11 functions as a controller (arithmetic unit) that controls the entire medical information processing device 1 by executing a program stored in the memory 12. The processor 11 is, for example, a processor such as a central processing unit (CPU), a graphics processing unit (GPU), or a tensor processing unit (TPU). The processor 11 may be composed of a plurality of processors. The processor 11 is an example of a computer.

The memory 12 is configured by various volatile memories and non-volatile memories such as a random access memory (RAM), a read only memory (ROM), and a flash memory. Further, the memory 12 stores a program for executing processing executed by the medical information processing device 1. Note that a part of information stored in the memory 12 may be stored by one or a plurality of external storage devices capable of communicating with the medical information processing device 1, or may be stored by a storage medium detachable from the medical information processing device 1.

The interface 13 is one or more interfaces for electrically connecting the medical information processing device 1 and another device. These interfaces may be a wireless interface such as a network adapter for wirelessly transmitting and receiving data to and from another device, or may be a hardware interface for connecting to another device via a cable or the like.

Note that the hardware configuration of the medical information processing device 1 is not limited to the configuration illustrated in FIG. 2. For example, the medical information processing device 1 may include at least one of the input device 2 or the output device 3.

### (3) Medical Document Generation Processing

Next, details of medical document generation processing executed by the medical information processing device 1 will be described. Schematically, the medical information processing device 1 generates a prompt including medical entities acquired from the target medical record and text of the target medical record, the text being relevant to the medical entities, and executes generation of a medical document with an LLM by using the prompt. As a result, the medical information processing device 1 generates a medical document in which a relationship between medical entities is also suitably considered.

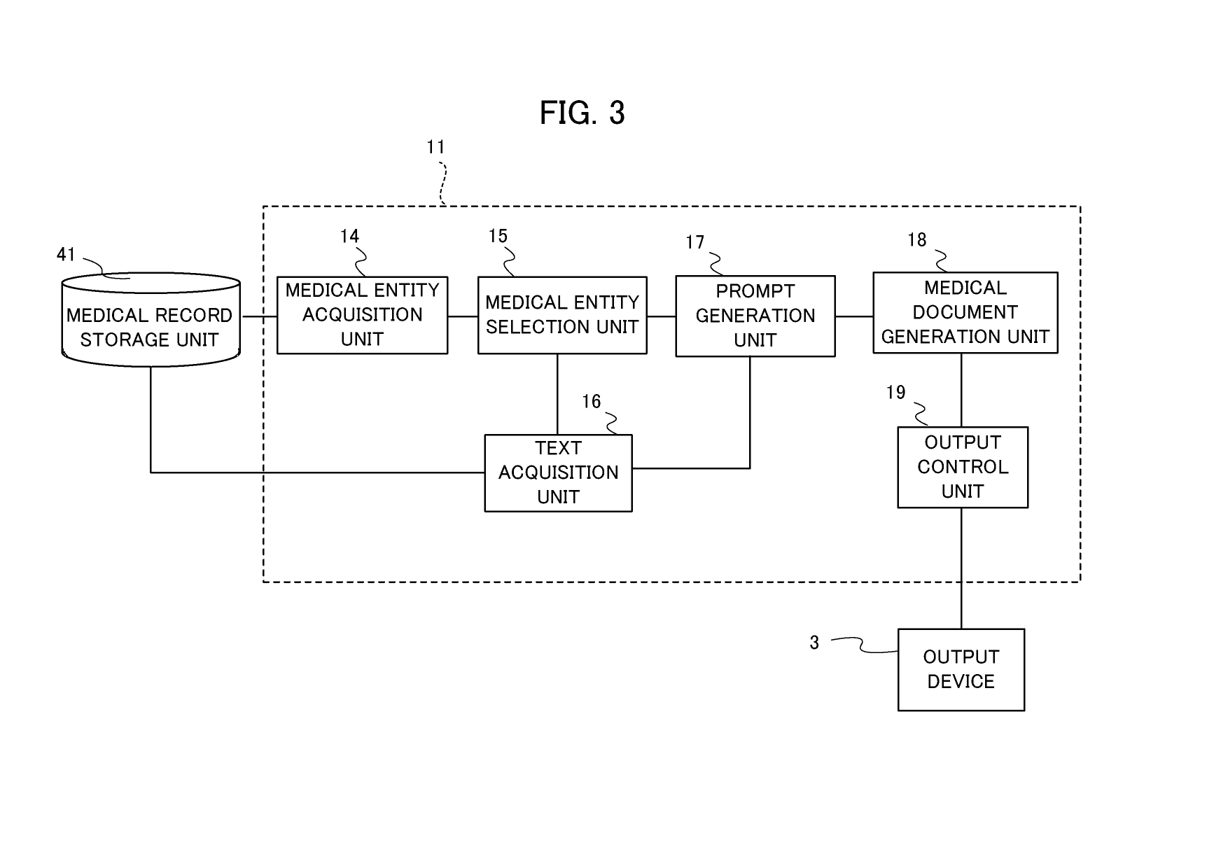

FIG. 3 is an example of functional blocks of a processor 11 of the medical information processing device 1. The processor 11 of the medical information processing device 1 functionally includes a medical entity acquisition unit 14, a medical entity selection unit 15, a text acquisition unit 16, a prompt generation unit 17, a medical document generation unit 18, and an output control unit 19. Note that, here, while blocks that exchange data with each other are connected by a solid line, a combination of the blocks that exchange data with each other is not limited to this. The same applies to diagrams of other functional blocks described later.

The medical entity acquisition unit 14 acquires the target medical record identified by an input or the like by the input device 2, from the medical record storage unit 41 via an interface 13, acquires the medical entities from the acquired target medical record, and supplies the acquired medical entities to the medical entity selection unit 15.

In this case, the medical entity acquisition unit 14 first extracts named entities related to medical care (for example, a treatment, an examination, a symptom, and the like) from the target medical record. The medical entity acquisition unit 14 determines a state as to whether each of the named entities is positive (yes or performed) or negative (no or not performed), and creates medical entities in which an expression representing the state is added to the named entity. For example, a medical entity (“coughing: yes”) is created by combining characters representing a state of a symptom (for example, “yes”) with a named entity (for example, “coughing”) representing the symptom. In another example, a medical entity (“○○ examination scheduled to be performed”) is created by combining characters indicating a state of an examination (for example, “scheduled to be performed”) with a named entity (for example, “○○ examination”) representing the examination. The above-described “to create” includes a mode of “to extract and classify” and also includes a mode of “to generate” using the LLM.

For example, the medical entity acquisition unit 14 may acquire the above-described medical entities by using a machine learning model trained in advance to extract a medical entity in a case where a document is input. In this case, the medical entity acquisition unit 14 inputs the target medical record to the above-described machine learning model and acquires the medical entities output by the machine learning model. Instead of this, the medical entity acquisition unit 14 may execute processing such as extraction of the named entities by using named entity recognition technology of dictionary-based, rule-based, or the like, or may generate a medical entity with the LLM.

The medical entity acquisition unit 14 may further execute processing (that is, timeline) of identifying a time series of the acquired medical entities. A method for identifying a time relationship of named entities (entities) extracted from structured data and unstructured data is disclosed in, for example, JP 2005-508544 A. The medical entity acquisition unit 14 may acquire, from the target medical record, medical entities associated with date and time information by using a machine learning model (including a language model such as the LLM) trained to output relevant date and time together with the medical entities.

The medical entity selection unit 15 selects, from the medical entities acquired by the medical entity acquisition unit 14, a medical entity to be used for creating the medical document, and supplies the selected medical entity to the text acquisition unit 16 and the prompt generation unit 17. In this case, for example, the medical entity selection unit 15 may select the medical entity based on statistical information of a medical entity included in a medical document created in the past. For example, the statistical information is information indicating frequency of each medical entity included in the medical document created in the past, and the medical entity selection unit 15 selects a medical entity whose frequency is equal to or more than a predetermined degree, from among medical entities acquired by the medical entity acquisition unit 14. In this case, for example, a table listing medical entities whose frequency is equal to or more than the predetermined degree may be stored in the storage device 4, and the medical entity selection unit 15 may select a medical entity included in the table, from among the medical entities acquired by the medical entity acquisition unit 14. The table may be prepared in advance for each type of medical document to be generated. That is, because medical entities selected for each type of medical documents such as a referral and a discharge summary are different, a table may be created and held for each type of the medical documents. In this case, the medical entity selection unit 15 selects a medical entity with reference to a table associated with the type of the medical document to be generated. In another example, the medical entity selection unit 15 may select the medical entity to be used for generating the medical document, by using a machine learning model. In this case, the machine learning model selects a medical entity necessary for generating the medical document from among input medical entities, and outputs the selected medical entity. The machine learning model may be the LLM. In still another example, the medical entity selection unit 15 may selectably display, with the output device 3, the medical entities acquired by the medical entity acquisition unit 14, and select a medical entity specified by a user input. In this case, the medical entity selection unit 15 may selectably display the medical entities acquired by the medical entity acquisition unit 14 side by side in time series (for example, in association with relevant dates) by the output device 3, and highlight the medical entity selected by the user input.

The text acquisition unit 16 acquires text of the target medical record relevant to the medical entity selected by the medical entity selection unit 15, and supplies the acquired text to the prompt generation unit 17. Here, the “text” represents a sentence in which the target medical record is divided by a predetermined rule (for example, a line feed and a period). For example, when the medical entity acquisition unit 14 extracts the named entities from the target medical record, the medical entity acquisition unit 14 stores a correspondence between the extracted named entities and text in the target medical record to which the named entities belong. Then, the text acquisition unit 16 refers to the correspondence described above and identifies text to which the named entities included in the medical entities selected by the medical entity selection unit 15 belong. In another example, by using the LLM, the text acquisition unit 16 may extract, from the target medical record, text relevant to the medical entities selected by the medical entity selection unit 15. In this case, the text acquisition unit 16 includes, in a prompt, the target medical record and the medical entity selected by the medical entity selection unit 15, and inputs, to the LLM, the prompt to instruct extraction of the text of the target medical record including the medical entity. As a result, the text acquisition unit 16 acquires text output as an answer by the LLM.

The prompt generation unit 17 generates a prompt based on the text acquired by the text acquisition unit 16 and the medical entity selected by the medical entity selection unit 15, and supplies the generated prompt to the medical document generation unit 18.

In this case, for example, the storage device 4 or the like stores fixed phrase information representing a fixed phrase of a prompt in which portions for inputting text and a medical entity are identified, and the prompt generation unit 17 generates the prompt in which the above-described text and the above-described medical entity is applied to the fixed phrase represented by the fixed phrase information.

In another example, the prompt generation unit 17 may generate the prompt by using the LLM. A prompt to instruct the LLM to generate a medical document is also referred to as a “first prompt”, and a prompt for generating the first prompt with the LLM is also referred to as a “second prompt”. In this case, the prompt generation unit 17 generates the second prompt to instruct to generate the first prompt, by using the text and medical entity described above. In this case, for example, the prompt generation unit 17 generates the second prompt in which a sentence instructing to create a prompt for generating the medical document with the LLM and a sentence indicating that the above-described text and medical entity are used in generation of the medical document are included together with the above-described text and medical entity. Then, the prompt generation unit 17 acquires the first prompt that the LLM outputs by the generated second prompt being input to the LLM.

The medical document generation unit 18 generates a medical document based on the prompt generated by the prompt generation unit 17. In this case, the medical document generation unit 18 acquires the medical document that the LLM outputs by the prompt generated by the prompt generation unit 17 being input to the LLM. The medical document generation unit 18 supplies the generated medical document to the output control unit 19.

The output control unit 19 causes the output device 3 to display the medical document generated by the medical document generation unit 18, and receives a correction input of the medical document by the input device 2. In this case, for example, the output control unit 19 displays a sentence indicated by the medical document, in an input field in which text editing is possible. After that, the output control unit 19 may perform processing of storing, in the storage device 4, the memory 12, or the like, the medical document generated by the medical document generation unit 18 or a medical document corrected by the received correction input, transmitting the medical document to another device, or printing the medical document with a printer.

In a case where the generated medical document is applied to a document (for example, a referral, a medical certificate, a discharge summary, or the like) having a predetermined format, the output control unit 19 may further correct the medical document in such a way that the medical document fits in an entry field for filling out the medical document in the document. For example, in a case where the number of characters that can be input in the above entry field is set, the medical document is further summarized in such a way as to be within the number of characters. In this case, the output control unit 19 may summarize the medical document by using a summarization method (for example, summarization by the LLM).

The output control unit 19 may cause the output device 3 to display an input screen that receives an input of information identifying the medical record for which the medical document is generated, and receive, with the input device 2, a user input for identifying the medical record on the input screen. In this case, the output control unit 19 supplies an input signal input by the input device 2 to the medical entity acquisition unit 14.

Here, each component of the medical entity acquisition unit 14, the medical entity selection unit 15, the text acquisition unit 16, the prompt generation unit 17, the medical document generation unit 18, and the output control unit 19 can be implemented by, for example, the processor 11 executing a program. Each component may also be achieved by recording a necessary program in an optional nonvolatile storage medium and installing the program as necessary. At least a part of these components is not limited to be achieved by software by a program, and may be achieved by a combination of any of hardware, firmware, and software, or the like. At least a part of these components may be achieved using, for example, a user-programmable integrated circuit such as a field-programmable gate array (FPGA) or a microcontroller. In this case, a program including the above components may be achieved by using the integrated circuit. At least a part of the components may include an application specific standard produce (ASSP), an application specific integrated circuit (ASIC), or a quantum processor (quantum computer control chip). In this manner, the components may be achieved by various types of hardware. The same applies to other example embodiments described later. These components may also be achieved by, for example, cooperation of a plurality of computers by using a cloud computing technology or the like.

Next, a specific example of the medical document generation processing will be described.

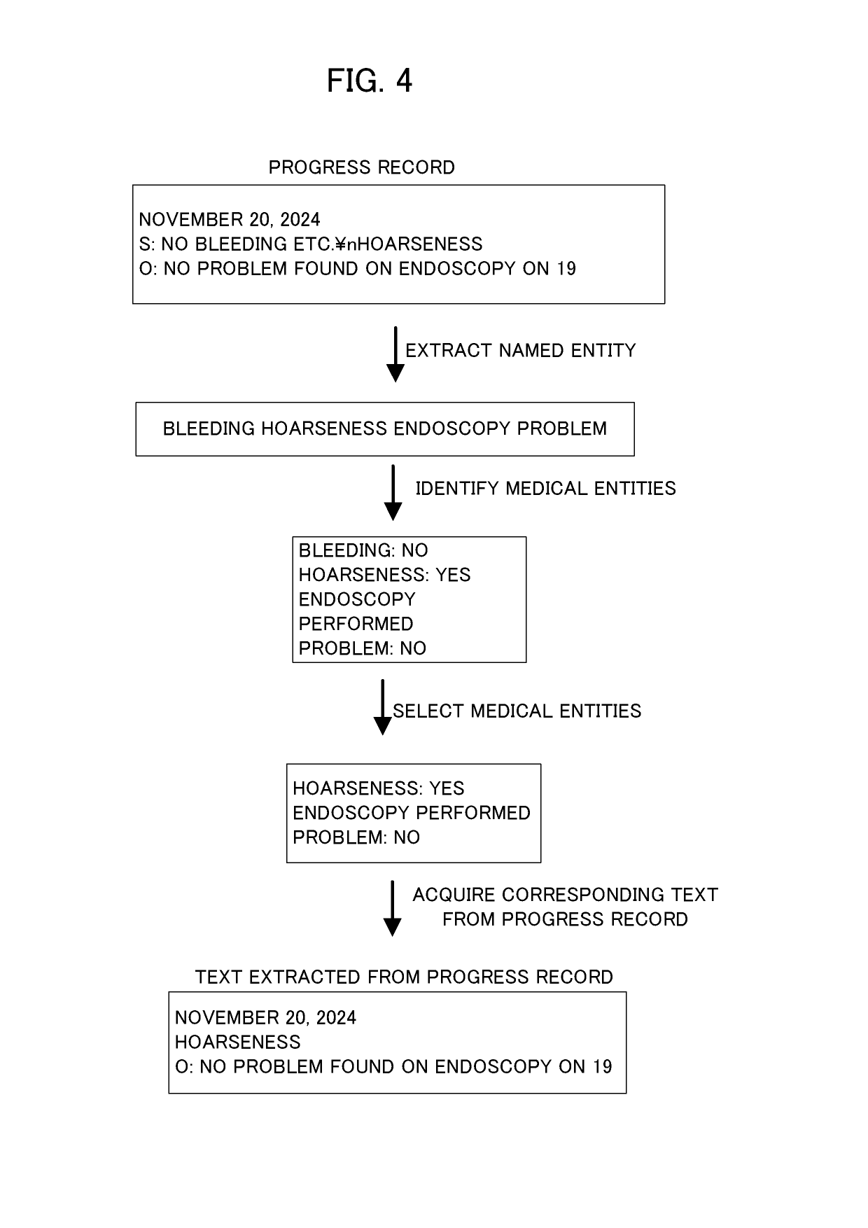

FIG. 4 is a diagram illustrating a flow of processing executed by the medical entity acquisition unit 14, the medical entity selection unit 15, and the text acquisition unit 16 in FIG. 3 in a specific example in which the medical document generation processing is performed with a progress record as the target medical record. The progress record illustrated in FIG. 4 includes four pieces of text of “November 20, 2024”, “S: no bleeding etc.”, “hoarseness”, and “O: no problem found on endoscopy on 19”. “\n” represents a line feed, “S” is a symbol representing “subjective information”, and “O” is a symbol representing “objective information”.

In this case, first, the medical entity acquisition unit 14 extracts four named entities “bleeding”, “hoarseness”, “endoscopy”, and “problem” from the progress record, and identifies a medical entity based on these named entities. Here, the medical entity acquisition unit 14 estimates each of states of the four named entities from the progress record, and identifies four medical entities “bleeding: no”, “hoarseness: yes”, “endoscopy performed”, and “problem: no” generated by adding words representing the estimated states to the relevant named entities.

Thereafter, the medical entity selection unit 15 selects medical entities important in generating the medical document (here, a discharge summary), from among the four medical entities by using statistical information or the like. Here, the medical entity selection unit 15 selects the three medical entities other than “bleeding: no”, which are “hoarseness: yes”, “endoscopy performed”, and “problem: no”.

The text acquisition unit 16 acquires, from the progress record, text relevant to the three medical entities selected by the medical entity selection unit 15. Here, the text acquisition unit 16 acquires the three pieces of text of “November 20, 2024”, “hoarseness”, and “O: no problem found on endoscopy on 19” from the progress record. Here, as an example, the text acquisition unit 16 acquires the text “November 20, 2024” indicating date and time, regardless of the medical entities selected by the medical entity selection unit 15. As described above, the text acquisition unit 16 may unconditionally extract text indicating date and time, regardless of a medical entity selected by the medical entity selection unit 15.

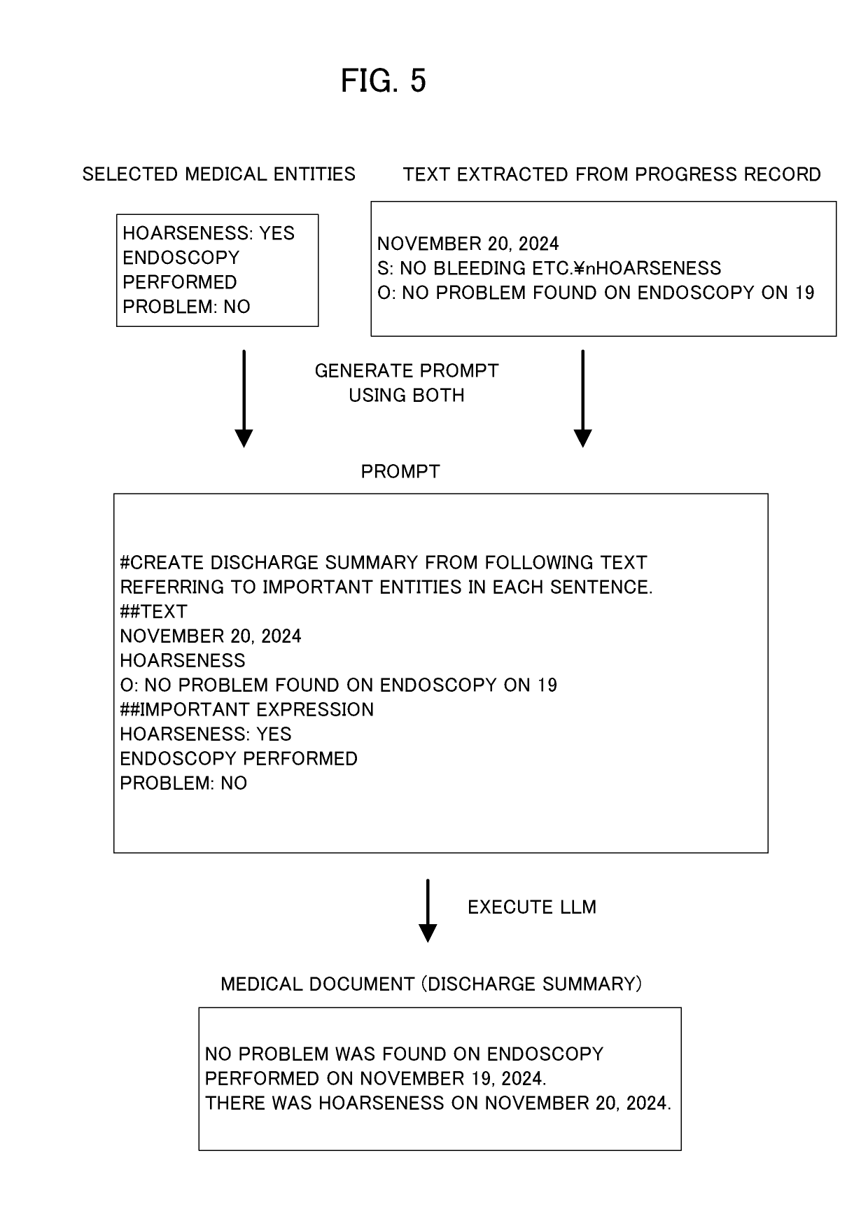

FIG. 5 is a diagram illustrating a flow of processing executed by the prompt generation unit 17 and the medical document generation unit 18 in FIG. 3 in the specific example of the medical document generation processing. The prompt generation unit 17 generates a prompt including the medical entities selected by the medical entity selection unit 15 and the text extracted by the text acquisition unit 16. Here, a fixed phrase “#Create discharge summary from following text. Refer to important expressions appearing in each sentence also.” instructing generation of a discharge summary by using medical entities selected by the medical entity selection unit 15 and text extracted by the text acquisition unit 16 is provided, the text extracted by the text acquisition unit 16 is provided after a fixed phrase “##text ”, and the medical entities selected by the medical entity selection unit 15 are provided after a fixed phrase"##important expression".

The medical document generation unit 18 acquires the medical document (here, the discharge summary) that the LLM outputs by the prompt generated by the prompt generation unit 17 being input to the LLM. In this case, a discharge summary including “No problem was found on endoscopy performed on November 19, 2024.” and “There was hoarseness on November 20, 2024.” is generated. As described above, the discharge summary that is an accurate summary of the progress record accurately reflecting relationships between the medical entities is generated.

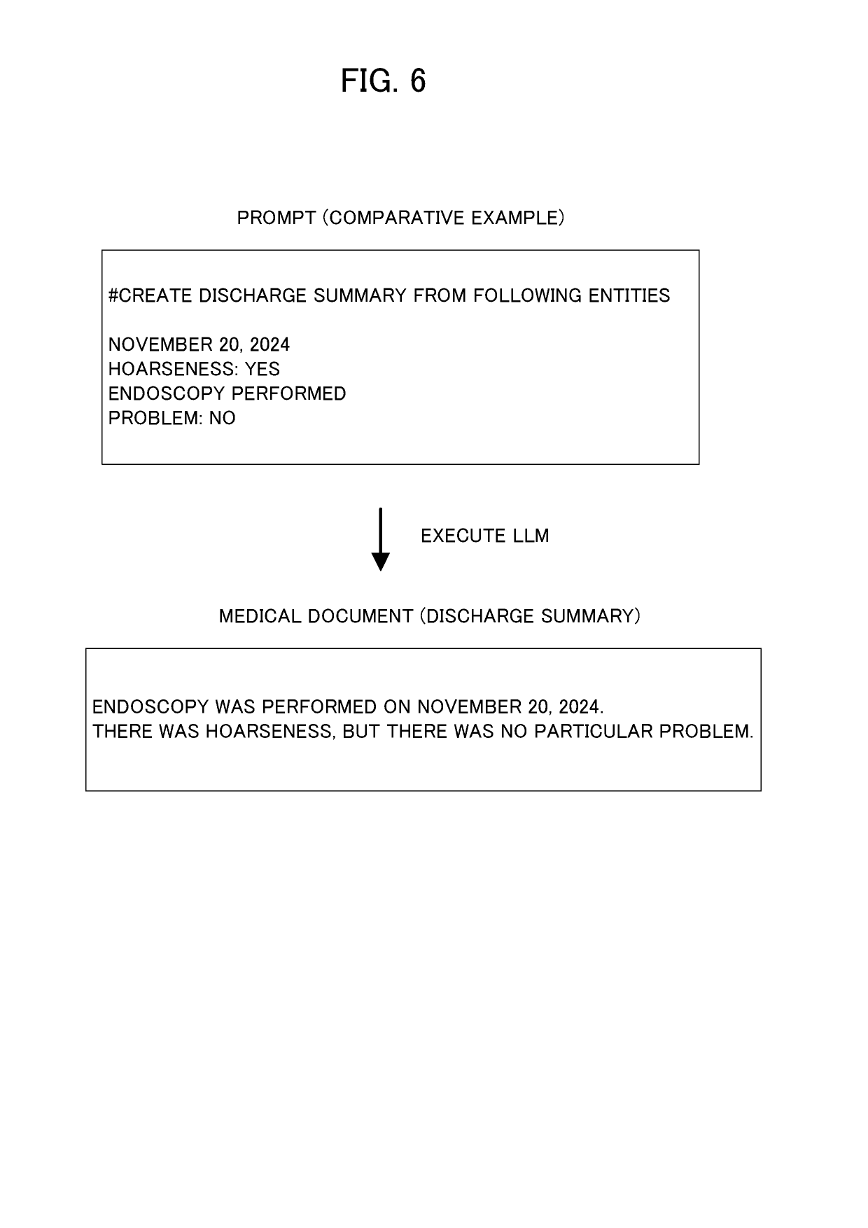

Here, a comparative example in which the text of the progress record is not included in a prompt will be described. FIG. 6 illustrates the prompt in the comparative example and a medical document generated by the prompt. In the comparative example, a prompt for instructing generation of the discharge summary by identifying the medical entities selected by the medical entity selection unit 15 and the text “November 20, 2024” in the progress record indicating date and time is generated. The medical document (discharge summary) output by the LLM by using such a prompt includes an erroneous sentence “Endoscopy was performed on November 20, 2024.”. In practice, it is described in the progress record that the endoscopy is performed on 19. In addition, a vague sentence “There was hoarseness, but there was no particular problem” is also included in the generated medical document. The progress record actually indicates that there is no problem as a result of the endoscopy. As described above, if a medical document is generated only based on medical entities, relationships between the medical entities may not be accurately considered, and a hallucination may occur.

In consideration of the above, the medical information processing device 1 includes text of the progress record including the medical entities in the prompt, in addition to the important medical entities. Thus, the discharge summary that is an accurate summary of the progress record accurately reflecting relationships between the medical entities can be generated.

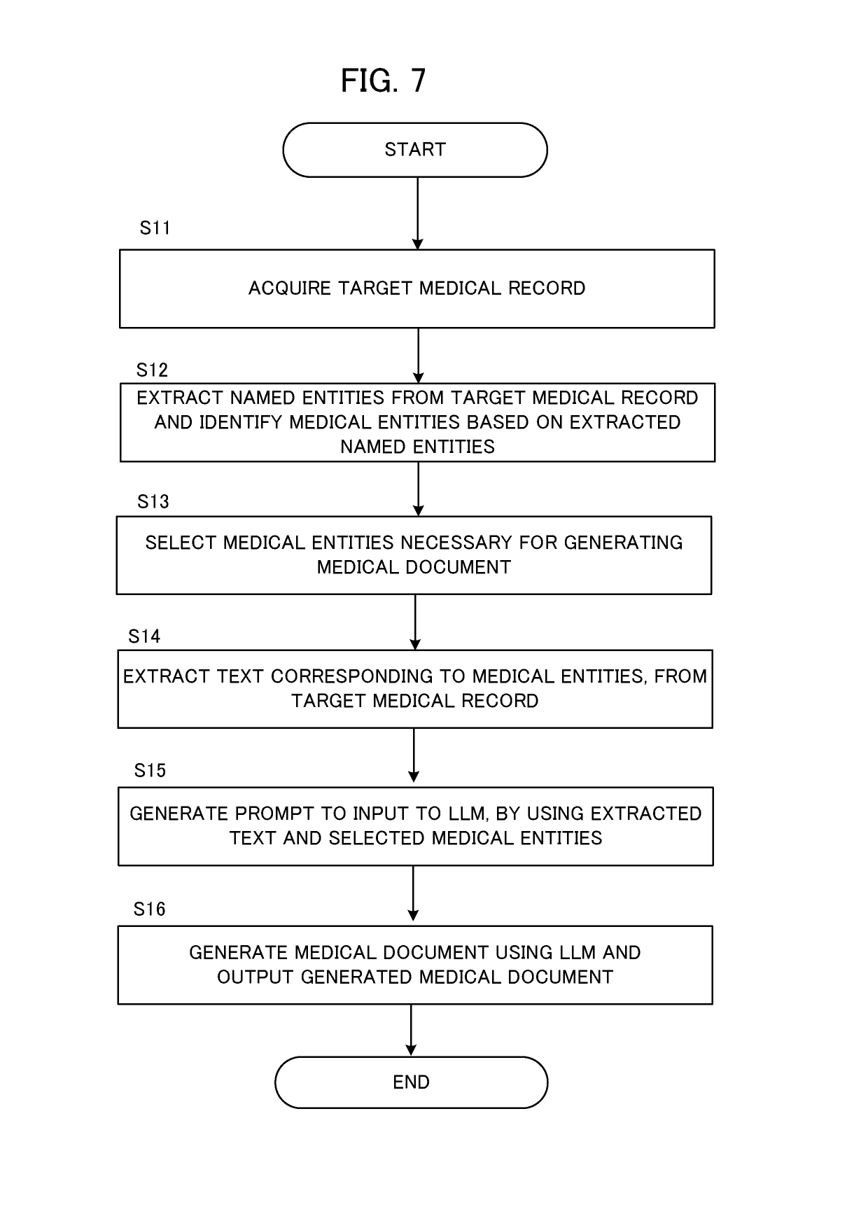

FIG. 7 illustrates an example of a flowchart executed by the medical information processing device 1. For example, the medical information processing device 1 executes processing of the flowchart illustrated in FIG. 7 upon detecting a request for generating a medical document with a designation of a target medical record.

First, the medical information processing device 1 acquires the target medical record (step S11). In this case, for example, the medical information processing device 1 extracts a medical record of the patient, which is specified by a user input or the like, from the medical record storage unit 41 as the target medical record.

Next, the medical information processing device 1 extracts named entities from the target medical record and identifies medical entities based on the extracted named entities (step S12). Then, the medical information processing device 1 selects a medical entity necessary for generating the medical document from among the medical entities identified in step S12 (step S13). Then, the medical information processing device 1 extracts text relevant to the medical entity, from the target medical record (step S14).

Next, the medical information processing device 1 generates a prompt to input to the LLM, by using the text extracted in step S14 and the medical entity selected in step S13 (step S15). Then, the medical information processing device 1 generates the medical document output by the LLM in response to input of the prompt generated in step S15, and outputs the generated medical document (step S16). In this case, for example, the medical information processing device 1 displays the medical document with the output device 3, receives a correction input of the medical document by the input device 2, and corrects the medical document in such a way as to fall within an entry field of a document to be applied. The medical information processing device 1 may store a finally generated medical document in the storage device 4, the memory 12, or the like, may transmit the medical document to another device, or may print the medical document with a printer.

### Second Example Embodiment

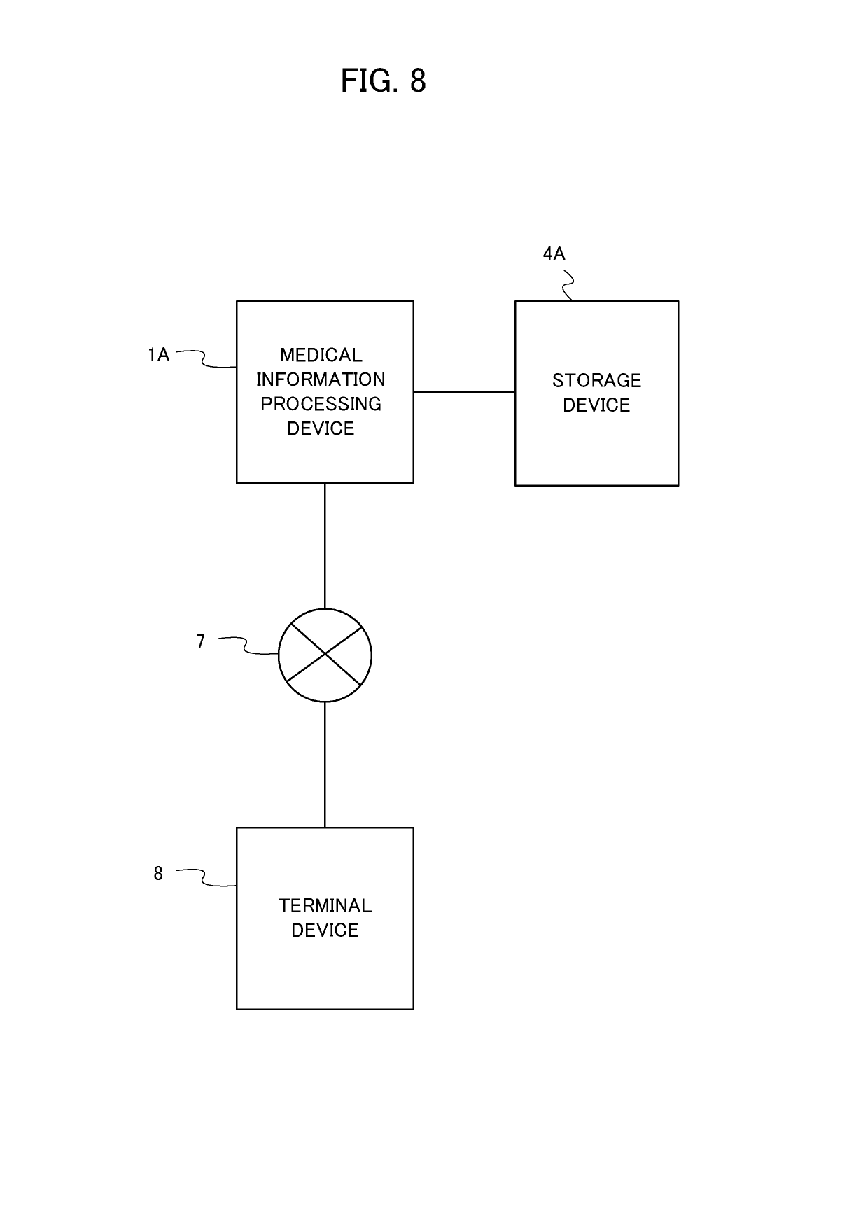

FIG. 8 illustrates a schematic configuration of a medical information processing system 100A. The medical information processing system 100A according to a second example embodiment is a server client model system, and a medical information processing device 1A that functions as a server device performs the processing of the medical information processing device 1 in the first example embodiment. Hereinafter, the same components as those of the first example embodiment are appropriately denoted by the same reference signs, and the description thereof will be omitted.

The medical information processing system 100A mainly includes the medical information processing device 1A that functions as a server, a storage device 4A that stores data similar to the data stored in the storage device 4 according to the first example embodiment, and a terminal device 8 that functions as a client. The medical information processing device 1A and the terminal device 8 perform data communication via a network 7.

The terminal device 8 is a terminal having an input function, a display function, and a communication function, and functions as the input device 2 and the output device 3 illustrated in FIG. 1. The terminal device 8 may be, for example, a personal computer, a tablet terminal, a personal digital assistant (PDA), and the like. The terminal device 8 transmits an input signal or the like based on a user input to the medical information processing device 1A.

The medical information processing device 1A has the same hardware configuration and functional blocks as those of the medical information processing device 1 according to the first example embodiment. The medical information processing device 1A then receives the input signal and the like from the terminal device 8 via the network 7, identifies a target medical record and the like based on the received information, and generates a medical document based on the identified target medical record, and the like. The medical information processing device 1A exchanges an output signal indicating information or the like related to a generation result of the medical document and an input signal indicating information or the like related to correction of the generated medical document with the terminal device 8 via the network 7. That is, in this case, the terminal device 8 functions as the input device 2 and the output device 3 in the first example embodiment.

As described above, the medical information processing system 100A in the second example embodiment can generate a medical document relevant to a target medical record identified by a user and present the medical document to the user.

### Third Example Embodiment

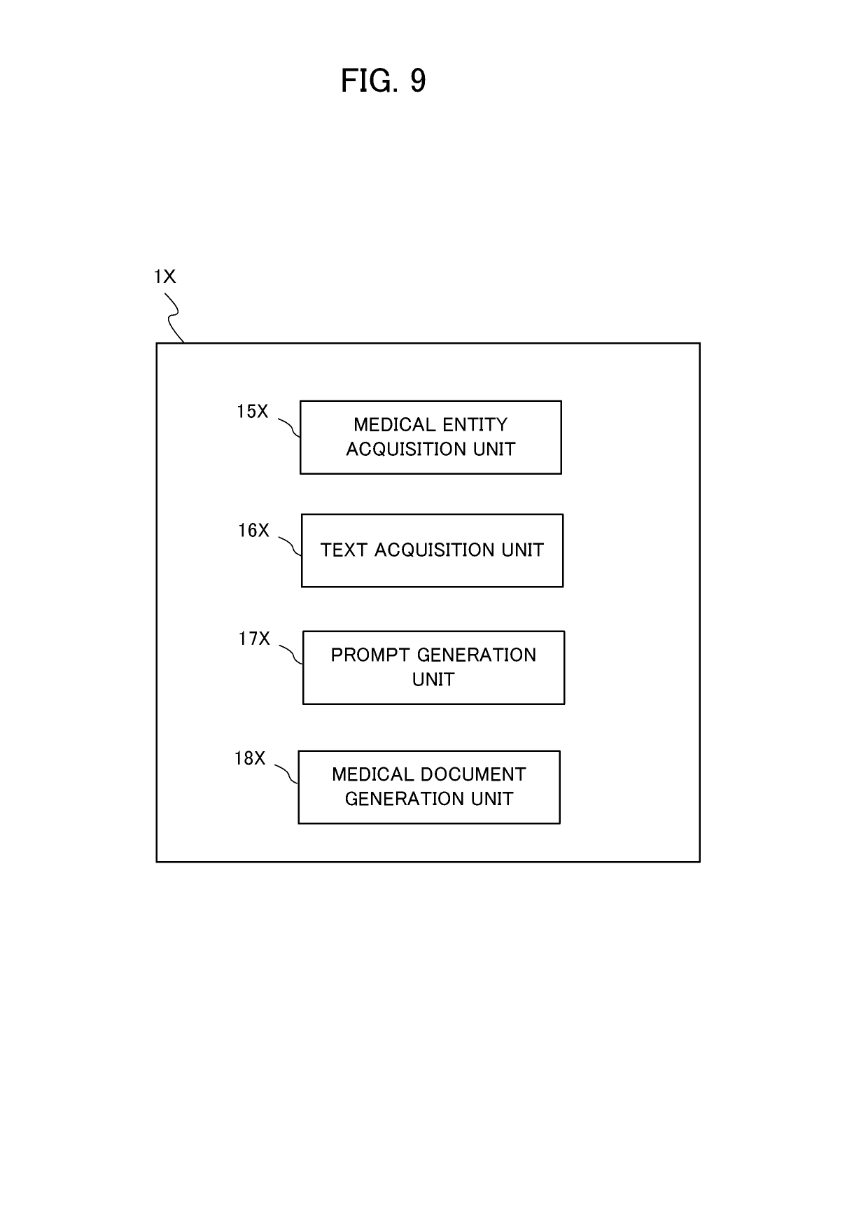

FIG. 9 is a block diagram of a medical information processing device 1X. The medical information processing device 1X includes a medical entity acquisition unit 15X, a text acquisition unit 16X, a prompt generation unit 17X, and a medical document generation unit 18X. The medical information processing device 1X may be composed of a plurality of devices.

The medical entity acquisition unit 15X acquires a medical entity included in a medical record. The medical entity acquisition unit 15X may be, for example, the medical entity acquisition unit 14 and the medical entity selection unit 15 in the first example embodiment and the second example embodiment.

The text acquisition unit 16X acquires text corresponding to the medical entity from the medical record. The text acquisition unit 16X may be, for example, the text acquisition unit 16 in the first example embodiment and the second example embodiment.

The prompt generation unit 17X generates a prompt for instructing a language model to generate a medical document, the prompt including the medical entity and the text, wherein the language model is trained to output, upon receiving a prompt, an answer to the prompt. The prompt generation unit 17X may be, for example, the prompt generation unit 17 in the first example embodiment and the second example embodiment.

The medical document generation unit 18X generates the medical document based on the prompt and the language model. The medical document generation unit 18X may be, for example, the medical document generation unit 18 in the first example embodiment and the second example embodiment.

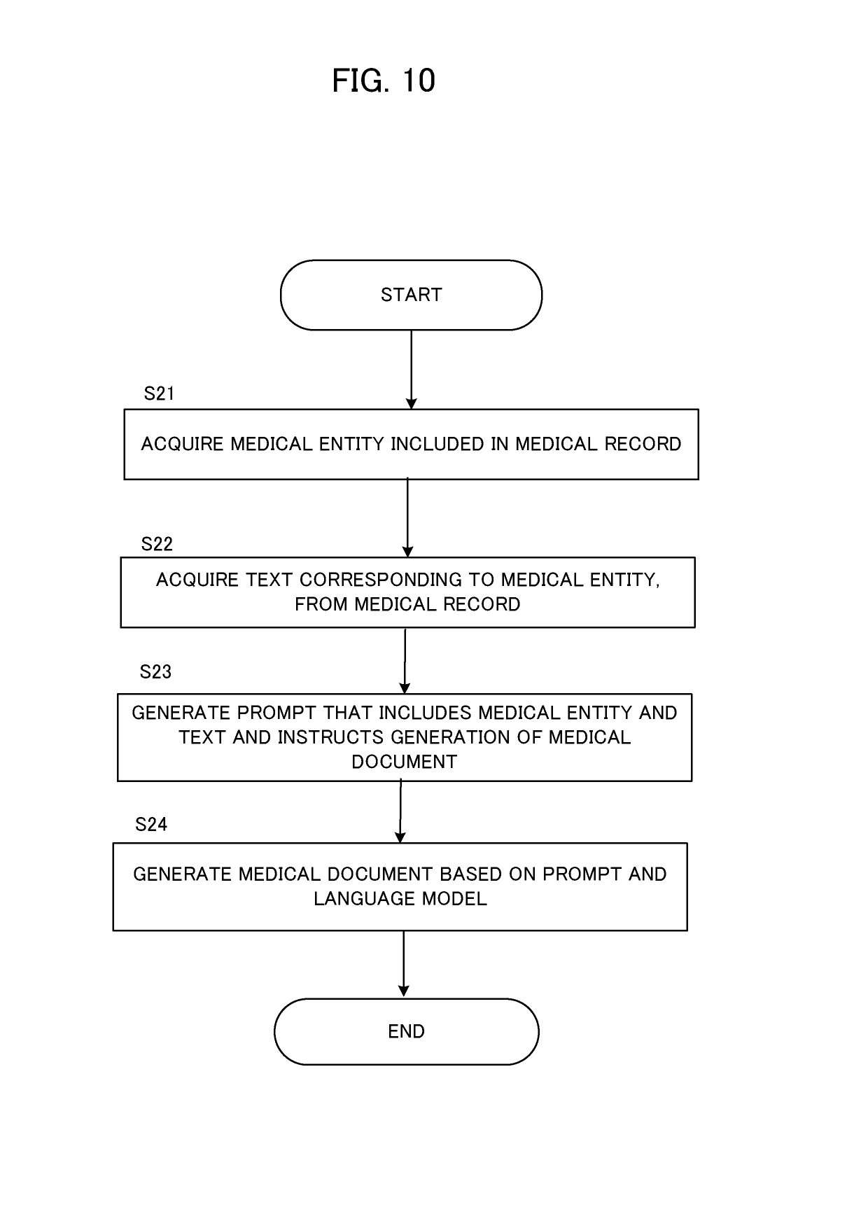

FIG. 10 is an example of a flowchart showing a processing procedure executed by the medical information processing device 1X. The medical entity acquisition unit 15X acquires a medical entity included in a medical record (Step S21). The text acquisition unit 16X acquires text corresponding to the medical entity from the medical record (Step S22). The prompt generation unit 17X generates a prompt for instructing a language model to generate a medical document, the prompt including the medical entity and the text (Step S23). The medical document generation unit 18X generates the medical document based on the prompt and the language model (Step S24).

According to the third example embodiment, the medical information processing device 1X makes it possible to generate an accurate medical document from a medical record.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

In addition, part or all of the above-described example embodiments (including modifications thereof; the same applies hereinafter) may also be described as the following Supplementary Notes, but the present disclosure is not limited thereto. Further, part or all of the configurations described in the Supplementary Notes dependent on Supplementary Note 1 may also depend on Supplementary Notes 9 and 10 in the same dependent manner as the Supplementary Notes dependent on Supplementary Note 1. Furthermore, without being limited to the apparatuses, methods, and storage media described in the Supplementary Notes, part or all of the configurations described as the Supplementary Notes may similarly be applied, without departing from the scope of the above-described example embodiments, to methods, various types of hardware, software, various recording media (including storage media) for recording software, or systems.

### Supplementary Note 1

A medical information processing device comprising:
a medical entity acquisition means for acquiring a medical entity included in a medical record;
a text acquisition means for acquiring text corresponding to the medical entity, from the medical record;
a prompt generation means for generating a first prompt for instructing a language model to generate a medical document, wherein the first prompt includes the medical entity and the text and wherein the language model is trained to output, upon receiving a prompt, an answer to the prompt; and
a medical document generation means for generating the medical document based on the first prompt and the language model.

### Supplementary Note 2

The medical information processing device according to Supplementary Note 1, wherein the prompt generation means generates, based on the medical entity and the text, a second prompt to be input to the language model to generate the first prompt, and acquires the first prompt output by the language model in response to the second prompt.

### Supplementary Note 3

The medical information processing device according to Supplementary Note 1, wherein the medical entity acquisition means selects the medical entity to be used for generation of the first prompt from medical entities included in the medical record, based on statistical information related to medical entities included in medical documents generated in the past, or based on a machine learning model.

### Supplementary Note 4

The medical information processing device according to Supplementary Note 1, wherein the medical entity acquisition means extracts a named entity related to medical care from the medical record, and generates the medical entity by adding, to the named entity, an expression representing a state of the named entity.

### Supplementary Note 5

The medical information processing device according to Supplementary Note 1, wherein the text is a sentence that includes the medical entity and is delimited by at least a period or a line break.

### Supplementary Note 6

The medical information processing device according to Supplementary Note 1, wherein the text acquisition means acquires the text output by the language model in response to the medical record.

### Supplementary Note 7

The medical information processing device according to Supplementary Note 1, further comprising: an output control means for outputting the medical document.

### Supplementary Note 8

The medical information processing device according to Supplementary Note 7, wherein the output control means displays the medical document in a mode of receiving an input for correcting the medical document, or stores or prints a document in which the medical document is contained in an entry field.

### Supplementary Note 9

A method executed by a computer, comprising:
acquiring a medical entity included in a medical record;
acquiring text corresponding to the medical entity, from the medical record;
generating a first prompt for instructing a language model to generate a medical document, wherein the first prompt includes the medical entity and the text and wherein the language model is trained to output, upon receiving a prompt, an answer to the prompt; and
generating the medical document based on the first prompt and the language model.

### Supplementary Note 10

A non-transitory computer readable storage medium storing a program executed by a computer, the program causing the computer to:
acquire a medical entity included in a medical record;
acquire text corresponding to the medical entity, from the medical record; generate a first prompt for instructing a language model to generate a medical document, wherein the first prompt includes the medical entity and the text and wherein the language model is trained to output, upon receiving a prompt, an answer to the prompt; and
generate the medical document based on the first prompt and the language model.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. Each example embodiment can be appropriately combined with other example embodiments. All Patent and Non-Patent Literatures mentioned in this specification are incorporated by reference in its entirety.

1, 1A, 1X Medical information processing device
2 Input device
3 Output device
4 Storage device
8 Terminal device
100, 100A Medical information processing system

1. A medical information processing device comprising:
at least one memory configured to store instructions; and
at least one processor configured to execute the instructions to:
acquire a medical entity included in a medical record;
acquire text corresponding to the medical entity, from the medical record;
generate a first prompt for instructing a language model to generate a medical document, wherein the first prompt includes the medical entity and the text and wherein the language model is trained to output, upon receiving a prompt, an answer to the prompt; and
generate the medical document based on the first prompt and the language model.

2. The medical information processing device according to claim 1,
wherein the at least one processor is configured to execute the instructions to
generate, based on the medical entity and the text, a second prompt to be input to the language model to generate the first prompt, and
acquire the first prompt output by the language model in response to the second prompt.

3. The medical information processing device according to claim 1,
wherein the at least one processor is configured to execute the instructions to select the medical entity to be used for generation of the first prompt from medical entities included in the medical record, based on statistical information related to medical entities included in medical documents generated in the past, or based on a machine learning model.

4. The medical information processing device according to claim 1,
wherein the at least one processor is configured to execute the instructions to
extract a named entity related to medical care from the medical record, and
generate the medical entity by adding, to the named entity, an expression representing a state of the named entity.

5. The medical information processing device according to claim 1,
wherein the text is a sentence that includes the medical entity and is delimited by at least a period or a line break.

6. The medical information processing device according to claim 1,
wherein the at least one processor is configured to execute the instructions to acquire the text output by the language model in response to the medical record.

7. The medical information processing device according to claim 1,
wherein the at least one processor is configured to further execute the instructions to output the medical document.

8. The medical information processing device according to claim 7,
wherein the at least one processor is configured to execute the instructions to display the medical document in a mode of receiving an input for correcting the medical document, or store or print a document in which the medical document is contained in an entry field.

9. A method executed by a computer, comprising:
acquiring a medical entity included in a medical record;
acquiring text corresponding to the medical entity, from the medical record;
generating a first prompt for instructing a language model to generate a medical document, wherein the first prompt includes the medical entity and the text and wherein the language model is trained to output, upon receiving a prompt, an answer to the prompt; and
generating the medical document based on the first prompt and the language model.

10. A non-transitory computer readable storage medium storing a program executed by a computer, the program causing the computer to:
acquire a medical entity included in a medical record;
acquire text corresponding to the medical entity, from the medical record;
generate a first prompt for instructing a language model to generate a medical document, wherein the first prompt includes the medical entity and the text and wherein the language model is trained to output, upon receiving a prompt, an answer to the prompt; and
generate the medical document based on the first prompt and the language model.
